(19) United States
(12) Patent Application Publication
(10) Pub. No.: US 2006/0293708 A1
(43) Pub. Date: Dec. 28, 2006

(54) Device for the treatment of bones and/or soft parts of the human or animal body and/or for the modification of cells and tissues by means of extra-corporal shock waves

(76) Inventor: Axel Voss, Kreuzlingen (CH)

Correspondence Address: ARNOLD & FERRERA, L.L.P., 2401 FOUNTAIN VIEW DRIVE, SUITE 630, HOUSTON, TX, 77057, US

(21) Appl. No.: 11/474,232

(22) Filed: Jun. 23, 2006

Related U.S. Application Data
(63) Continuation-in-part of application No. PCT/EP04/14692, filed on Dec. 23, 2004.

(30) Foreign Application Priority Data
Dec. 23, 2003 (DE) 103 60 942.3

(51) Int. Cl. A61B 17/00

(52) U.S. Cl. 606/201

(57) ABSTRACT

The invention relates to a shock wave for the treatment of bones and/or soft parts of the human or animal body and/or for the modification of cells and tissues as well as the generation of apoptosis, in particular through mechanical stress (shearing forces) exerted on cells.

### STATEMENT OF RELATED CASES

This is a continuation-in-part of International Application PCT/EP2004/014692, with an international filing date of Dec. 23, 2004, still pending.

### FIELD OF THE INVENTION

The invention relates to a device for treating bones and/or soft parts of the human or animal body and/or for modifying cells and tissues by means of extra-corporal shock waves. Furthermore, the invention relates to a shock wave for treating the illnesses described below.

### BACKGROUND

In the literature L. Gerdesmeyer et al.: ‘Physikalisch-technische Grundlagen der extrakorporalen Stoβwellentherapie’ in Orthopäde 2002; 31: pp. 610-617, various methods are described for generating shock waves and applying them in destroying kidney stones and cystoliths as well as in the field of orthopaedics. Further indications in treating bones and soft parts of the human or animal body are given by Beat Dubs in ‘Extra-corporal shock wave therapy (ESWT): a new achievement or only a placebo’ Swiss med. forum, No. 9, 26 Feb. 2002.

In the prior art until now, conventional shock waves are applied, which begin with a positive pressure peak, which follows a low frequency negative pressure phase (tensile wave) with reduced amplitude. These shock waves have worked satisfactorily in destroying kidney stones, cystoliths and suchlike and at present, are still controversially discussed as a form of treatment for other indications.

The present invention relates to the use of shock waves for the improved treatment of inflammatory and non-inflammatory bone indications and soft part indications as well as the modification of cells and tissues.

In particular, the invention relates to the use of shock waves for exerting mechanical stress (shearing forces) on cells, wherein their apoptosis is implemented. This happens for example by means of an initiation of the ‘death receptor pathway’ and/or the cytochrome c-pathway and/or a caspase cascade.

The term apoptosis is understood to refer to the initiation of a genetically controlled program, which leads to the ‘cell suicide’ of individual cells in the tissue formation. As a result, the cells concerned and their organoids shrink and disintegrate into fragments, the so-called apoptotic bodies. These are phagocytized afterwards by macrophages and/or adjoining cells.

The occupation of receptors with certain messenger material, the withdrawal of growth factors, inter-cell contact, DNA damage, metabolism or cell cycle disturbances, cytotoxic t-cells, belong for example to the internal and external negative signals which can release apoptosis as well as increased levels of oxidants in the cell or mutagenic agents. Members of the necrosis factor (TNF) and lymphotoxin tumour family which bind both to the TNF receptor as well as the CD95 ligand, which is homologous to the necrosis factor tumour and binds to the receptor protein CD95 (older descriptions: Fas, APO1), relate particularly to the signal proteins which bind to cell surface receptors.

Protein p53 for example, is an important regulator existing in mammalian cells, which among other things, checks the intactness of DNA. If damage is irreparable, p53 arranges cell death by inducing the synthesis of the apoptosis-promoting bax protein. If an external death signal meets a cell, then the authorization of this instruction is checked by the proteins of the bcl 2 family. The various members of this family interconnect themselves to dimers, which act to promote or restrain the apoptosis. Some of these proteins are bound to membranes of the endoplasmatic reticulum, membranes of the core and membranes of the mitochondrions. They form pores and in this way can influence the ion permeability of the membranes. As a consequence, mitochondrial proteins such as cytochrome c cross over into the cytoplasm and promote the implementation of the apoptosis.

Furthermore, in the course of apoptosis, induction of cellular renovation and repair mechanisms takes place by diffusing heat shock proteins (hsp), such as the members of the hsp70 family (Hsp70, Hsp72 etc.) for example, which among other things, take part in protein folding and in protein transport as well as in re-constructing the folding of denatured proteins.

Now surprisingly, it has been ascertained that administered shock waves act reciprocally with biological tissue in such a manner that certain cell reactions, such as for example apoptosis, are released.

The cell organoids, for the most part very sensitive elements and components of the cell, are in their biological equilibrium, exposed to very large forces in shortest time through extreme pressure changes of several hundred bar within the cell, i.e. shearing forces.

While one side of a cell organoid (e.g. mitochondrion) is already exposed, for example, to the maximum pressure of approx. 300 bar, the opposite (contra lateral) part of it remains physically still unaffected. Normal pressure prevails on this part of the cell organoid. Now, a shock wave passes through a complete cell within a few microseconds and at every point, subjects it to a maximum pressure change within a few nanoseconds.

In its course moreover, each shock wave covers both a positive pressure portion as well as a ‘negative’ tensile portion, which can likewise amount to 100 bar and, temporally staggered, takes place only a few microseconds (1-4 μs) after the ‘positive’ wave portion. Expressed in dimensions of length, this means: The positive pressure wave rise lasts approx. 1-10 ns. This corresponds to a distance of 1.5 μm to 15 μm at a speed of sound in water of approx. 1500 m/s.

As a comparison, the dimensions of some cell contents: mitochondrions: 0.5-2 μm, cell membrane: 5-10 nm, cells: 10-100 μm, cell core: 5-25 μm, core membrane: 20 nm and chromosomes: 1-10 μm.

In using shock waves according to the invention, the rising slope of the shock wave covers for example a distance, which a mitochondrion completely fulfils. Expressed differently: within its volume, an organoid with the size of a mitochondrion is exposed to an immense pressure change. This means an enormous mechanical stress both for the mitochondrion and the cell. In their dimension, the shearing forces arising with it lie in the range of several Newton.

The sloping flank of the shock wave has a time length of less than one microsecond. The pressure difference Δp arising with it, is up to 400 bar and means considerably greater mechanical stress for the individual cells. Forces of several Newton arise with it. The time length of these forces is however so short and the inertia of the mass of the cells so large that they do not necessarily tear the cell up. It is crucial for the biological effectiveness of the shock wave however, that certain threshold values are fulfilled, e.g. the pressure change/time, the maximum pressure and the number of pulses. For example, dp/dt (the temporal pressure change), plays as a result, a significant and determining role.

These forces do not conclusively lead to the destruction of the cells or cell components as the time intervals, in which these forces work, are too short and the mechanical inertia of the components does not allow for this. Due to its high amplitude, very short pulse duration and very short distance stretch of approx. 1.5 μm to 15 μm, a shock wave of the device according to invention, which is prepared as if it were a tensile wave, is however surprisingly suitable, for example, in reciprocally acting with or within mitochondrions, in chromosomes or membrane proteins.

If now for example, too many cell core chromosomes are damaged due to the reciprocal effect with the shock wave according to invention, numerous proteins are activated. Proteases (e.g. apoptotic proteases such as caspases) which destroy key cell proteins important for structure preservation, replication and repair of DNA and the new synthesis of proteins belong to these, as well as endonucleases (e.g. CAD or DNase I), which take part in breaking up chromatin.

Meanwhile, fourteen different caspases are known in humans, which are arranged in a signal cascade and are divided up into signal caspases, strengthener caspases and effector caspases. Important caspases are, besides caspase 9, caspase 3 and procaspase 8, the so-called inititiator caspase, which is activated by a ligand with an adapter molecule (FADD) andfor its part, activates caspase 3. As a result, this relates to the so-called apoptosis ‘death receptor pathway’. ‘Death receptors’ are membrane molecules from the TNF receptor family such as, amongst others, TNF a (tumour necrosis factor alpha). A series of cytoskeleton proteins belong to the most important substrates of caspase 3, which are important for maintaining the shape and motility of cells. Breaking up these proteins leads to dramatic changes in the morphology of the cell during the apoptosis.

The effect of the caspases is supplemented by endonucleases, which break up the chromatin of the cell in a characteristic way. In breaking up apoptotic chromatin, DNA is broken up in ranges which are not protected by association with histones, such that DNA fragments are formed by approximately 200 base pairs or multiples.

Due to the use of shock waves according to the invention however, cytochrome c, which binds to the APAF 1 protein, can also be released when some mitochondrions (0.5 μm to 2 μm) have been damaged or destroyed, which afterwards oligomerizes ATP dependently and through this, activates an initiator caspase, in this case caspase 9. This caspase 9 subsequently activates, similar to caspase 8 the effector caspases, essentially caspase 3.

In accordance with the invention therefore, shock waves are used to exert shearing forces on cells, such that their natural apoptosis is implemented without inflammatory side effects. This happens in a way wherein shearing forces initiate the cytochrome c pathway for example, and implement a caspase cascade. Caspases are central components for inducing and terminating cell death, the inactivating of which usually blocks the apoptosis. However shock waves, according to the invention, now stimulate the formation of caspase 3.

Furthermore, an increased distribution of heat shock proteins can also be stimulated due to the use of the device in accordance with the invention, consequently for example hsp70 interacts with the mitochondrial distribution of cytochrome c and can therefore suppress a certain form of the apoptosis, i.e. the NO induced apoptosis. The apoptosis initiated by the p53 protein however remains uninfluenced by it.

Shock waves according to the invention can be particularly advantageously used for treating necrotically modified regions and structures in muscle tissue, in particular heart muscle tissue, for stimulating cartilage growth in conjunction with arthritic illnesses of the joints, for initiating the differentiation of embryonic or adult stem cells in vivo and in vitro according to the surrounding cell formation, for treating tissue weaknesses, in particular cellulite and for breaking up collagen fibre producing cells and fat cells, as well as for activating growth factors, in particular TGF β.

Shock waves according to the invention can likewise be used for preventing edema formation and/or expansion, as well as for breaking up edemas, for treating ischemia, rheumatism, joint illnesses, for treating the jaw (paradontitis), cardiologic illnesses and cardiac infarcts, pareses (paralyses), inflammations of the nerve, cross section paralyses, arthrosis, arthritis, for prophylaxis of scar formation, for treating scar formation and/or nerve scarring, for treating achillodynia, achillobursitis and other bone necroses, as well as for treating head tumours in the region of the brain.

Further preferential areas of application lie in the treatment of inflammatory and non-inflammatory bone and soft part indications, in which according to invention shock waves function as genome stressors. They influence the metabolism of the cells, release tensile wave induced NO, promote revascularisation, induce reconstruction of hyalin cartilage (osteochondrosis dissecans) and induce natural apoptosis (non-necrotic cell death without inflammatory reaction).

Due to an improved healing process caused in this way, the shock waves according to invention are thus particularly suitable for treating scarred tendinuous and band tissue as well as badly healing open wounds, in particular ulcus cruris/hypertonicum, ulcus varicosum or ulcus terebrans as well as bum and large tissue defects.

A further use relates to the treatment of skin tissue areas which suffer hair loss due to natural causes, trauma, thermal, chemical related or other forces e.g. burns, chemical burns. According to the working mechanism described above hair follicle cells can be stimulated to start over to produce hair. The stimulated tissue or scar tissue exposed to shock waves can even be revived to grow missing hair follicle cells.

Shock waves also stimulate vascularisation of scare tissue and thereby initiate building of missing pigmentation.

A further use relates to the treatment of spinal marrow injuries and nerve injuries, for example spinal marrow injuries with accompanying oedemization.

Fundamentally, the use of shock waves according to the invention is appropriate in all cases of illnesses to do with degraded apoptoserates, preferably hepato-cellular cancers, cholangio cancers, colon cancers or pancreas cancers, particularly in cases of chemotherapy resistance.

In addition, shock waves according to the invention are suitable for treating tumours with disturbedprotein p53 expression. The loss of the p53 tumour suppressor gene function is for example, a frequent event in malignant tumours. The absence of the p53 dependent apoptosis not only plays an essential role in the carcinogenesis, but also in therapy resistance as well as in chemo-resistance and/or ray-resistance malignant tumours. Thus, despite typically non-mutated p53, human melanome and/or mesotheliome, for example, exhibits a distinct resistance against radiation and chemotherapy. The therapy resistance allows for the assumption of inactivation of certain molecular components in p53-associated apoptosis cascades. Mutation-conditioned inactivation of p53-inducing genes as well as inactivation of p53-induced effector genes could both be brought into question as possible causes for the therapy resistance which is observed.

Various cancer-releasing viruses have developed mechanisms, in order to prevent the death of their host cells. Humane Papilloma viruses (HPV) form protein E6 for example, which binds and inactivates the p53 apoptosis promoter. The Epstein Barr virus (EBV), which causes mononucleosis and Burkitt's lymphoma, produces a Bcl 2 similar protein, which prevents the apoptosis.

Furthermore, shock waves according to the invention are thus used for treating tumours caused by virus infections, e.g. for treating a cervix cancer.

The improved extra-corporal shock wave therapy produces an inverted shock wave, which delivers a negative pressure pulse as a peak value, i.e. prepared as a tensile wave. Acoustic energy introduced to such an extent generates high shearing stresses in biological tissue. At the same time, the inverted pressure pulse works as a tissue and genome stressor and is suited to stimulating regeneration processes. The negative pressure pulse lies, as a result, within a range of 10ˆ7 bar/second <|dp/dt|<10ˆ12 bar/second, if |dp/dt| designates the absolute value of the temporal pressure change, wherein the rise time Tᵣ lies within the range of 1 ns<Tᵣ<1000 ns, preferably within the range of 1 ns<Tᵣ<100 ns.

In a particularly favourable way, an inverted shock wave can be generated with an electro-hydraulic shock wave source, using for example an underwater spark gap, which at first delivers a primary shock wave which is inverted on a reflection element, or generated electromagnetically.

Preferably, the reflection element is designed as a paraboloid, an ellipsoid, and/or as a spherical reflector, designed in particular for grouping the shock waves at a focus, wherein the focus size lies in the range of 1 mm to 200 mm.

The reflection element is a soft reflector closed within itself. According to an embodiment it is designed as a solid reflector, made out of a whole material with a low E-module, which is so conditioned, that in order to invert the shock wave, said reflector, in comparison to the medium in which the shock wave is generated, must have low acoustic impedance Z in the range of 0.005 Mrayl<Z<0.5 Mrayl. Suitable materials for this are rubber, neoprene, latex or closed-porous foams. ‘Solid reflector’ and ‘whole material’ mean a material thickness, which is sufficient, in order to essentially absorb the transmitted portion of the incidental wave and in that manner suppress it, such that the reflector only brings about the pure phase reversal of the incidental pressure pulse.

In another embodiment, the reflection element comprises a coating, made again from one of the aforementioned materials with the E-module and impedance values indicated. In this case, the thickness of the layer can be set in such a way that the low frequency wave portions following-on the peak value of the inverted shock wave are compensated by superposing using wave portions which are at first transmitted and which are normally reflected on the reflector background, such that essentially, only the negative pressure pulse works.

The device for generating shock waves in accordance with the invention contains a self-contained reflector applicator head with a coupling membrane, which is connected flexibly and mobile by a lead to a supply and control unit. It is particularly favourable if the device contains a plug-in replaceable part which is made out of at least one electrical connection (plug) and a reflector part of the applicator head.

Further advantages, characteristics and possibilities for application result from the following description of the invention.

### BRIEF DESCRIPTION OF THE DRAWINGS

FIG. 1: an overview of the device according to invention

FIG. 2a: a schematic representation of the pressure course of a normal shock wave

FIG. 2b: an inverted shock wave

FIG. 3: an electro-hydraulic shock wave source with a coated reflector

FIG. 4: electro-hydraulic shock wave source with a solid soft reflector

### DETAILED DESCRIPTION OF THE DRAWINGS

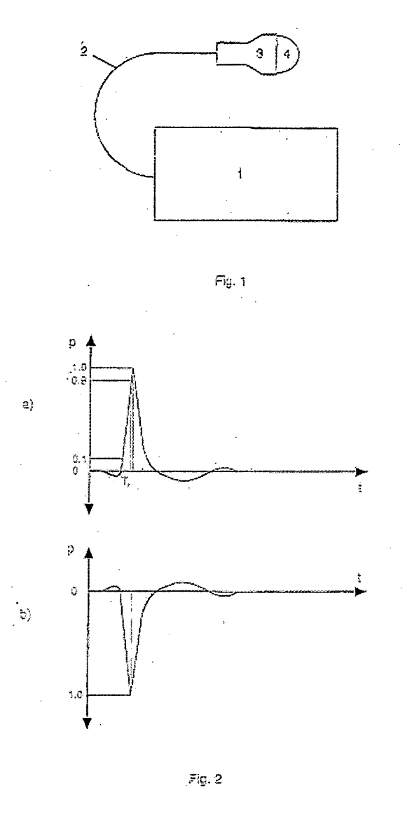

The essential elements of the device for treating bones and/or soft parts of the human or animal body and/or for modifying cells and tissues by means of extra-corporal shock waves are schematically arranged in FIG. 1. The device contains an applicator head 3 with a coupling membrane 4, which is connected flexibly and mobile by a lead 2 to a supply and control unit 1. The applicator head 3 is adjusted onto the object to be worked on or the point of a human or animal body which is to be treated.

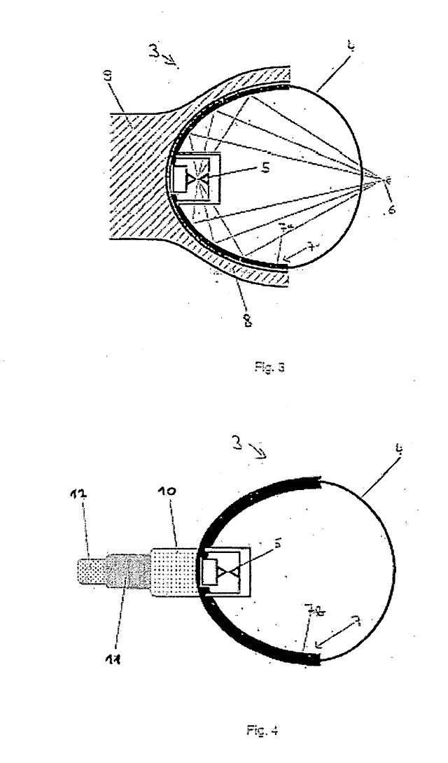

FIG. 2a shows schematically the temporal pressure course of a normal shock wave. Shock waves are transient pressure fluctuations, which spread in all directions. In each case, they begin with a rapid rise in pressure, wherein the peak pressure is reached within 1 ns to 1000 ns. A longer persisting, negative pressure phase with clearly smaller amplitude follows the positive pressure pulse. This so-called tensile wave portion is usually regarded as unfavourable as, due to arising tensile forces, the cohesion forces of the medium concerned can be easily overstepped and cavitation effects caused. The wave rise is usually described by rise time Tr and temporal pressure change dp/dt in the range between 10% and 90% of the peak pressure.

FIG. 2b shows the inverted shock wave, as it is used in accordance with the present invention. At the peak value, it supplies a negative pressure impulse and as a result, a high tensile wave portion, followed by a positive, longer-wave positive pressure wave portion.

Although the biological effect of inverted shock waves is possibly not yet understood in all detail, one can nevertheless assume that the high shearing forces which arise lead to stress features in the relevant cells in the tissue and possibly lead to a stimulation of the uric acid cycle or to the release of NO in biological tissue, wherein regeneration processes are started which are connected with tissue renewal.

FIG. 3 shows a sectional view through a water-filled applicator head 3 according to the electro-hydraulic principle. The elliptical reflector 7 generates a focus 6 upon which the shock waves gather. Depending upon the application range, the reflector can also be designed as a paraboloid or spherical surface, without it generating a defined focus.

In the sketched embodiment, the electrodes 5 are located at the internal focal point of the elliptical reflector 7. The reflector has a coating made from Latex 7a, which on the surface, exhibits a distinctly smaller acoustic impedance Z than water. In a known way, Z is calculated as a product of the speed of sound C and the density ρ, wherein the speed of sound, is again essentially determined by the E-module. Further suitable materials are rubber, neoprene and closed-porous foams. In crossing-over from the medium of water, with comparatively high acoustic impedance, into the coating, the shock wave experiences a phase reversal, wherein a large part of the acoustic energy is reflected in the form of the inverted shock wave. A lesser part is transmitted and, among certain absorption losses, is in the end reflected again. The thickness of the coating is, at the same time, selected in such a way with regard to distance stretch and absorption, that the normally reflected portion compensates the inverted pressure pulse following-on part of the shock wave. The reflector is located in a plastic holder 8 in the hand piece 9.

In the embodiment of FIG. 4, the coated reflector of FIG. 3 is exchanged for a solid soft reflector 7b, which essentially consists of whole rubber. It is dimensioned such that transmitted portions of the incidental shock wave are sufficiently suppressed by absorption such that the reflector brings about a pure phase reversal of the incidental shock wave. The device contains a plug-in replaceable part which is made out of at least one electrical connection and a reflector part of the applicator head 3. At the same time, the electrical connection contains a plug with a phase conductor 10, insulator 11 and internal conductor 12.

Reference Signs

FIG. 1:

1 supply and control unit
2 lead
3 applicator head
4 coupling membrane
   FIG. 2:
a) normal shock wave (course of pressure/time)
b) inverted shock wave (course of pressure/time)
   FIG. 3:
3 applicator head
4 coupling membrane
5 electrodes
6 focus (focus of the therapy)
7 reflector
7a surface coating made from latex
8 plastic holder for the reflector
9 hand piece
   FIG. 4:
3 applicator head
4 coupling membrane
5 electrodes
7 reflector
7b solid, soft or hard reflector made e.g. from latex
10 phase conductor
11 insulator
12 internal conductor

1. Shock wave for the modification of cells and tissues and the generation of apoptosis, in particular through mechanical stress (shearing forces) exerted on cells.

2. Shock wave as claimed in claim 1 for the treatment of tissues of the human or animal body, in particular ulcers (open wounds) of the epithelium (skin), necrotically modified regions and structures in muscle tissue, injuries of nervous tissue or connective tissue weaknesses.

3. Shock wave as claimed in claim 2 for the treatment of ulcus cruris/hypertonicum, ulcus varicosum or ulcus terebrans.

4. Shock wave as claimed in claim 2 for the treatment of burns, chemical burns and skin tissue defects.

5. Shock wave as claimed in claim 2 for the treatment of scar tissue.

6. Shock wave as claimed in claim 2 for the treatment of hair producing cells, hair follicle and hair loss.

7. Shock wave as claimed in claim 2 for the treatment of collagen fibre producing cells and fat cells.

8. Shock wave as claimed in claim 2 for the treatment of necrotically modified regions and structures in heart muscle tissue.

9. Shock wave as claimed in claim 2 for the treatment of cardiologic illnesses and cardiac infarcts.

10. Shock wave as claimed in claim 2 for the treatment of scarred tendinuous and band tissue.

11. Shock wave as claimed in claim 2 for the treatment of scar formation or nerve scarring.

12. Shock wave as claimed in claim 2 for the treatment of spinal marrow injuries or nerve injuries.

13. Shock wave as claimed in claim 2 for the treatment of paralyses, in particular cross section paralyses.

14. Shock wave as claimed in claim 2 for the treatment of inflammation of nerves.

15. Shock wave as claimed in claim 2 for the treatment of ischemia.

16. Shock wave as claimed in claim 2 for the treatment of cellulite.

17. Shock wave as claimed in claim 2 for the treatment of edemas.

18. Shock wave as claimed in claim 2, wherein the shock wave has a negative pressure pulse at the peak value.

19. Shock wave as claimed in claim 14, wherein the negative pressure pulse lies within a range of the absolute value |dp/dt| of the temporal pressure change of 10ˆ7 bar/second <|dp/dt|<10ˆ12 bar/second and a rise time Tᵣ of the rising slope of the negative pressure pulse of 1 ns <Tᵣ<1000 ns, preferably within the range of 1 ns<Tᵣ<100 ns.

20. Shock wave for the treatment of bones and joints of the human or animal body, in particular inflammatory and non-inflammatory bone indications or arthritic illnesses of the joints.

21. Shock wave as claimed in claim 20 for the treatment of the jaw (paradontitis).

22. Shock wave as claimed in claim 20 for the treatment of achillodynia, achillobursitis or other bone necroses.

23. Shock wave as claimed in claim 20, wherein the shock wave has a negative pressure pulse at the peak value.

24. Shock wave as claimed in claim 23, wherein the negative pressure pulse lies within a range of the absolute value |dp/dt| of the temporal pressure change of 10ˆ7 bar/second <|dp/dt|<10ˆ12 bar/second and a rise time Tᵣ of the rising slope of the negative pressure pulse of 1 ns<Tᵣ<1000 ns, preferably within the range of 1 ns<Tᵣ<100 ns.

25. Shock wave for the treatment of tumors in the human or animal body, in particular tumors with disturbed protein p53 expression.

26. Shock wave as claimed in claim 25 for the treatment of head tumors in the region of the brain.

27. Shock wave as claimed in claim 25 for the treatment of hepato-cellular cancers, cholangio cancers, colon cancers or pancreas cancers, particularly in cases of chemotherapy resistance.

28. Shock wave as claimed in claim 25 for the treatment of tumors caused by virus infections, in particular for the treatment of cervix cancer.

29. Shock wave as claimed in claim 25, wherein the shock wave has a negative pressure pulse at the peak value.

30. Shock wave as claimed in claim 29, wherein the negative pressure pulse lies within a range of the absolute value |dp/dt| of the temporal pressure change of 10ˆ7 bar/second <|dp/dt|<10ˆ12 bar/second and a rise time Tᵣ of the rising slope of the negative pressure pulse of 1 ns<Tᵣ<1000 ns, preferably within the range of 1 ns<Tᵣ<100 ns.
